(19) United States
(12) Patent Application Publication
(10) Pub. No.: US 2019/0110725 A1
(43) Pub. Date: Apr. 18, 2019

(54) Method and Apparatus for Autonomous Minimally-Invasive Capillary Blood Extraction

(71) Applicants: Robert Wilkes, Calgary (CA); Gang Wang, Calgary (CA); Martin Mintchev, Calgary (CA); Orly Yadid-Pecht, Calgary (CA)

(72) Inventors: Robert Wilkes, Calgary (CA); Gang Wang, Calgary (CA); Martin Mintchev, Calgary (CA); Orly Yadid-Pecht, Calgary (CA)

(21) Appl. No.: 16/157,483

(22) Filed: Oct. 11, 2018

Related U.S. Application Data
(60) Provisional application No. 62/574,062, filed on Oct. 18, 2017.

(51) Int. Cl. A61B 5/151; A61B 5/15; A61B 5/00; A61B 5/157

(52) U.S. Cl. CPC A61B 5/15109; A61B 5/15115; A61B 5/157; A61B 5/150343; A61B 5/6807; A61B 5/150022

(57) ABSTRACT

The present invention provides method and apparatus for minimally-invasive capillary blood extraction comprising an apparatus capable of the harnessing the energy from frequent natural mammalian movements and then converting it into forces needed to execute skin lancing, specifically an insertion of a lancet in the body of the mammal. The apparatus consists of a rotational and a translational member which work together to effectively translate an angular displacement of the rotational element caused by mammalian movements to translational displacement of a sliding element at an angle to the plane which the force from the mammal was applied in. The ability of the invention to harness free energy and efficiently use that energy for actuating a lancet and facilitating blood extraction allows for the miniaturization of blood sampling technology into a wearable device.

### BACKGROUND OF THE INVENTION

### 1. Technological Field

The present invention relates to a method and its associated apparatus for autonomous minimally-invasive capillary blood extraction comprising a miniature in-plane lancing device for the purpose of lancing the skin and extracting a whole blood sample from capillaries below the surface of the skin.

### 2. Background

Blood extraction is an important medical practice. Blood samples extracted from patients can be sent to laboratories for tests which can determine physiological states, diseases and drug effectiveness. If a blood test requires only a tiny amount of blood, blood extraction can be performed by patients themselves at home using miniature devices. For example, Diabetes mellitus is a systemic disorder that results in elevated blood glucose levels due to insulin deficiency in the body and subsequently leads to many secondary complications (American Diabetes Association, 2012). Diabetes mellitus requires long-term treatment, the goal of which is to achieve optimal glucose monitoring and control with the long-term aim of decreasing the risk of vascular complications while minimizing daily glycemic variations (Hendriks, Brokken, Oomens, Baaijens, & Horsten, 2000). Current standards for blood glucose monitoring for diabetics rely on finger-prick testing performed by patients at home using a device manually operated by themselves (Penfornis, Personeni, & Borot, 2011). Though accurate in detecting blood glucose levels, finger-prick testing is painful and inconvenient. In a recent systematic review of clinical trials, it has been found that providing a distraction can reduce the acute pain felt by children and adolescents during needle-related procedures (Uman, Chambers, McGrath, & Kisely, 2008). Therefore, users would feel less procedural pain if they were distracted by their ordinary daily activities. There is an obvious need for wearable blood extraction devices which would be pre-programmed to automatically obtain static blood samples multiple times over a long period of time with minimal pain and limited user intervention.

### 3. Description of Related Technologies

Standard diabetic monitoring relies on finger-prick testing by a miniature device (Penfornis, Personeni, & Borot, 2011). Though highly accurate in detecting blood glucose levels, finger-prick testing is painful and inconvenient. Therefore, patients, especially those in their youth and in active maturity, are often unable to adhere to the test schedule. As a result, irregular measurements limit the applicability of the finger-prick test and disturb the management of diabetes (Penfornis, Personeni, & Borot, 2011).

Continuous glucose monitoring (CGM), introduced in 1960s, is a concept which measures glucose levels in the interstitial fluid (ISF). A CGM device can be subcutaneously inserted and records ISF glucose level but only for 3-7 days (Penfornis, Personeni, & Borot, 2011). Clinicians rely on CGM to retrospectively understand glucose level trends within this short period and guide diabetes management (Oliver, Toumazou, Cass, & Johnston, 2009). Its accuracy is dependent on the equilibrium of glucose levels between ISF and blood. The balance between the two glucose levels further accounts for a time delay in the measurement (Penfornis, Personeni, & Borot, 2011) and requires frequent recalibration using finger-prick testing (Hoeks, Greven, & Valk, 2011).

There is an obvious need for wearable semi-invasive blood sampling devices which would be able to automatically obtain and analyze a series of static blood samples over an extended period of time with minimal pain and limited user intervention. This can be achieved by inserting a hypodermic needle to a level where capillaries are abundant but nerve endings are rare (Gattiker, Kaler, & Mintchev, 2005).

On average the skin of an adult has a thickness of roughly 2 mm (Martini, 2001). The outermost layer of the skin is the stratum corneum which is a thin but very dense and resistive compound of dead cells. The thickness of stratum corneum varies among different skin sites, ranging from 23.6±4.33 μm for the forearm and 173.0±36.96 μm for the palm (El-Laboudi, Oliver, Cass, & Johnston, 2013). Situated below is the epidermis which protects against the rays of the sun and has a thickness of 30 to 130 μm. The dermis, which is located under the epidermis and ranges 800-1500 μm in thickness, holds abundant blood vessels, hair follicles, sweat glands and few nerve endings (Hendriks, Brokken, Oomens, Baaijens, & Horsten, 2000). Lastly, the subcutaneous tissue is a fatty layer located below the dermis which connects to internal organs. It is usually about 1.2 mm deep (Hendriks, Brokken, Oomens, Baaijens, & Horsten, 2000). In order to successfully acquire blood sample, a lancet has to penetrate the resistive stratum corneum and reach the dermis layer which contains capillaries.

Prior methods of acquiring a blood sample suffer from the need for human intervention in order to control the lancing device. Kuhr and Forster U.S. Pat. No. 6,419,661 describe a lancing device for withdrawing blood for diagnostic purposes, which has been implemented in the Accu-chek blood glucose monitoring products by Roche. In this patent, a lancet holder for holding a lancet and a lancet drive having a loadable elastic drive spring are provided within an elongated housing. The triggering of the spring is performed by finger-pressing on a button. This device exemplifies the devices used in a standard finger-pricking test that require two separate components: a lancing device to perform the lancing as the first step and a blood testing device to load the blood sample as the second step. As described above, finger-prick testing suffers from low patient compliance due to painful experiences and inconveniences caused. Furthermore, time delays between these two steps may potentially contaminate blood samples and result in inaccurate tests.

Perez and Roe U.S. Pat. No. 8,257,276 describes a blood sampling assembly with a single spring-force triggered lancet and an integrated capillary-action based blood collection mechanism which can be directly associated with a blood testing device. This device is a convenient single disposable unit. Patients need to take only one action to get their blood glucose level reading. Other similar patents include Haynes U.S. Pat. No. 4,920,977 and Jordan et al., U.S. Pat. No. 4,850,973.

An automatic blood collection system comprising a plurality of lancets is also disclosed in Kelly, U.S. Pat. No. 6,530,892. A drive unit is implemented by a magnet, similarly to the above mentioned prior art. However, these types of integrated devices still require hand control and therefore the associated problems described before are still not overcome.

The difficulty of implementing a fully automated and wearable blood sampling and analysis device lies in the device miniaturization, and in particular, the actuator miniaturization. Maximal stroke and optimal insertion force are the two primary design factors for a lancing device for blood sampling. In order to penetrate the skin surface, the minimum lancing force required is approximately 30 gf (Tsuchiya, Nakanishi, Uetsuji, & Nakamachi, 2005). On the other hand, the actuator must be capable of moving the lancet to depths between 0.5 to 1.4 mm below the skin, where capillary vessels are abundant in order to successfully acquire blood samples. For manual lancing devices, a wide range of large actuators that can achieve these two requirements can be selected as there is relatively less restriction for the actuator size.

In order to provide sufficient lancing force and stroke for wearable blood sampling devices, other actuation methods have been proposed. Kaler et al. US Pat. App. No. 20050228313 proposed a miniature scale system that automatically penetrates the skin to extract a static blood sample for on-site analysis.

Sof-Tact™ is a fully integrated blood glucose testing device. The device features a lancet holder and a strip holder, which house a lancet and a test strip inside the device's cover. To perform a blood glucose test, users load a test strip and a lancet inside the device and then gently press the gasket on the forearm or other sites. Once a user presses the main button, the Sof-Tact™ electrical actuator automatically lances the skin, draws a blood sample using suction, transfers the sample to the embedded test strip and provides a glucose reading in 20 seconds.

The POGO™ System by Intuity Medical, Inc. (see http://www.presspogo.com/pogo/system/) is a commercially available blood glucose monitoring device that is designed to reduce the steps and components required for a finger-pricking test. The POGO™ System has an integrated cartridge which consists of multiple pairs of miniaturized lancet and test strip. The user presses a finger on the button of this device to trigger a skin-lancing mechanism below that button that pokes the fingertip and withdraws a blood sample, which is immediately wicked into a miniaturized strip to get a reading. At the end of each lancing, the cartridge revolves and reloads a new lancet and its associated strip for the next test.

It remains technically challenging to implement a fully automatic BGM device that can be wearable at the fingertips. As another point of view, such a device may not be accepted by the market as it is quite visible to others and will also affect users' hand activities. Furthermore, the fingertips are also abundant with nerve endings and are therefore sensitive to skin lancing. Automated skin lancing at the fingertips is expected to cause the same level of pain compared to manual fingerpricking, which could become a concern to potential users in the future. These semi-automated devices reduce errors due to mishandling related to the fingerpricking test. In spite of their advantages, semi-automated BGM devices are still bulky in size due to their mechanical design. Both Sof-Tact™ and Pogo™ are about the size of a cellphone and designed as handheld devices that require strong manual force input to trigger their skin lancing mechanisms. Therefore, they are not suitable for miniaturization towards a wearable device. The present invention provides a method and apparatus for using the forces exerted during normal mammalian movements to autonomously actuate a lancet to pierce the skin of the mammal which is integrated into a wearable autonomous device for ultimate convenience.

### 4. Prior References

### PATENT CITATIONS

| Patent No. | Title |
|---|---|
| 8,075,496 | Integrated device for diagnostic purposes |
| 8,641,643 | Sampling module device and method |
| 8,079,961 | Automatic biological analyte testing meter with integrated |
| | lancing device and methods of use |
| 7,604,592 | Method and apparatus for a point of care device |
| 7,415,299 | Monitoring method and/or apparatus |
| 7,967,752 | Sensor system as well as an arrangement and method for |
| | monitoring a constituent and in particular glucose in |
| | body tissue |
| 8,337,420 | Tissue penetration device |
| 8,679,033 | Tissue penetration device |
| 8,652,831 | Method and apparatus for analyte measurement test time |
| 8,641,643 | Sampling module device and method |
| 8,636,673 | Tissue penetration device |
| 8,574,895 | Method and apparatus using optical techniques to measure |
| | analyte levels |
| 8,496,601 | Methods and apparatus for lancet actuation |
| 8,435,447 | Integrated meter for analyzing biological samples |
| 8,414,503 | Methods and apparatus for lancet actuation |
| 8,360,991 | Tissue penetration device |
| 8,337,420 | Tissue penetration device |
| 8,157,748 | Methods and apparatus for lancet actuation |
| 8,079,960 | Methods and apparatus for lancet actuation |
| 8,075,496 | Integrated device for diagnostic purposes |
| 7,981,056 | Methods and apparatus for lancet actuation |
| 7,981,055 | Tissue penetration device |
| 7,862,520 | Body fluid sampling module with a continuous compression |
| | tissue interface surface |
| 7,850,622 | Tissue penetration device |
| 7,787,924 | Compact apparatus for noninvasive measurement of glucose |
| | through near-infrared spectroscopy |
| 7,378,007 | Combined lancet and electrochemical analyte-testing |
| | apparatus |
| 7,344,507 | Method and apparatus for lancet actuation |
| 7,175,642 | Methods and apparatus for lancet actuation |
| 7,133,710 | Compact apparatus for noninvasive measurement of glucose |
| | through near-infrared spectroscopy |
| 7,041,068 | Sampling module device and method |
| 7,025,774 | Tissue penetration device |
| 6,706,159 | Combined lancet and electrochemical analyte-testing |
| | apparatus |
| 5,971,941 | Integrated system and method for sampling blood and |
| | analysis |
| 5,487,748 | Blood Sampling Device |
| 4,995,402 | Medical droplet whole blood and like monitoring |
| 4,416,279 | Cappilary Blood Sampling Device |

### PATENT APPLICATION CITATIONS

| Patent App. No. | Title |
|---|---|
| 20080269639 | Device for obtaining bodily fluids for analysis |
| 20140121486 | Method and apparatus for analyte measurement test |
| | time |
| 20120264204 | Method and apparatus using optical techniques to |
| | measure analyte levels |
| 20120149999 | Tissue penetration device |
| 20120029385 | Insertion device systems and methods |
| 20110077478 | Body fluid sampling module with a continuous |
| | compression tissue interface surface |
| 20100324452 | Tissue penetration device |
| 20100286563 | Integrated Meter for Analyzing Biological Samples |
| 20090270765 | Integrated meter for analyzing biological samples |
| 20090209883 | Tissue penetrating apparatus |
| 20090138032 | Tissue penetration device |
| 20090131829 | Tissue penetration device |
| 20080294068 | Body fluid sampling module with a continuous |
| | compression tissue interface surface |
| 20080287831 | Methods and apparatus for lancet actuation |
| 20080262388 | Puncturing System For Collecting Body Fluid Sample |
| 20080214956 | Methods and apparatus for lancet actuation |
| 20080214917 | Method and apparatus for analyte measurement test |
| | time |
| 20080210574 | Method and apparatus for analyte measurement test |
| | time |
| 20080194989 | Methods and apparatus for lancet actuation |
| 20070276290 | Tissue Penetrating Apparatus |
| 20070244499 | Methods and apparatus for lancet actuation |
| 20070219572 | Integrated device for diagnostic purposes |
| 20070219463 | Methods and apparatus for lancet actuation |
| 20070173741 | Tissue penetration device |
| 20070142748 | Tissue penetration device |
| 20070064516 | Methods and apparatus for lancet actuation |
| 20060241667 | Tissue penetration device |
| 20060204399 | Method and apparatus using optical techniques to |
| | measure analyte levels |
| 20060195133 | Tissue penetration device |
| 20060195132 | Tissue penetration device |
| 20060195131 | Tissue penetration device |
| 20060195047 | Sampling module device and method |
| 20050011759 | Combined lancet and electrochemical analyte-testing |
| | apparatus |
| 20040049219 | Methods and apparatus for lancet actuation |
| 20040010207 | Self-contained, automatic transcutaneous physiologic |
| | sensing system |
| 20030212424 | Method and apparatus for lancet actuation |
| 20030191415 | Integrated sample testing meter |
| 20030187338 | Analyte monitoring device and methods of use |
| 20030083686 | Tissue penetration device |
| 20030083685 | Sampling module device and method |
| 20020130042 | Combined lancet and electrochemical analyte-testing |
| | apparatus |

### PAPER CITATIONS

American Diabetes Association. (2012). Standards of Medical Care in Diabetes—2012. Diabetes Cares, 35(2), S11-63.
El-Laboudi, A., Oliver, N. S., Cass, A., & Johnston, D. (2013). Use of Microneedle Array Devices for Continuous Glucose Monitoring: A Review. Diabetes Technology & Therapeutic, 15(1), 101-115.
Fruhstorfer, H., Muller, T., & Scheer, E. (1995). Capillary blood sampling: How much pain is necessary? Part 2: Relation between penetration depth and puncture pain. Practical Diabetes International, 12(4), 184-185.
Gattiker, G., Kaler, K., & Mintchev, M. (2005). Electronic Mosquito: designing a semi-invasive Microsystem for blood sampling, analysis and drug delivery applications. Microsyst Technol, 12(1), 44-51.
Hendriks, F. M., Brokken, D., Oomens, C. W., Baaijens, F. P., & Horsten, J. B. (2000). Mechanical Properties of Different Layers of Human Skin. Eindhoven: Philips Research Laboratories.
Hoeks, L., Greven, W., & Valk, H. d. (2011). Real-time continuous glucose monitoring system for treatment of diabetes: a systematic review. Diabet Med, 28(2), 386-94.
Martini, F. (2001). Fundamentals of Anatomy & Physiology. Upper Saddle River, N.J.: Prentice Hall.
Oliver, N., Toumazou, C., Cass, A., & Johnston, D. G. (2009). Glucose sensors: a review of current and emerging technology. Diabet Med, 26(3), 197-210.
Penfornis, A., Personeni, E., & Borot, S. (2011). Evolution of Devices in Diabetes Management. Diabetes Technology and Therapeutics, 13(4), S93-101.
Tsuchiya, K., Nakanishi, N., Uetsuji, Y., & Nakamachi, E. (2005). Development of Blood Extraction System for Health Monitoring System. Biomedical Microdevices, 7(4), 347-353.
Uman, L. S., Chambers, C. T., McGrath, P. J., & Kisely, S. (2008). A Systematic Review of Randomized Controlled Trials Examining Psychological Interventions for Needle-related Procedural Pain and Distress in Children and Adolescents: An Abbreviated Cochrane Review. Journal of Pediatric Psychology, 33(8), 842-854.

### OBJECTIVE OF THE INVENTION

The objective of the present invention is to disclose a method and its associated apparatus for minimally-invasive skin penetration comprising a miniature in-plane lancing device for the purpose of lancing the skin and extracting whole blood sample from capillaries below the surface of the skin.

The present invention aims to be integrated with a wearable blood sampling device so that blood sampling can be done without any human intervention.

### SUMMARY OF THE INVENTION

A method and its associated apparatus for minimally-invasive skin penetration and whole blood extraction is described. The proposed method may be implemented on a wearable blood sampling device which would be pre-programmed to automatically extract static blood samples multiple times over a prolonged period of time with minimal pain and limited user intervention. Whole blood samples collected using the present invention can be automatically analyzed on site for monitoring certain blood components.

According to one aspect of the invention, there is provided a system for fluid sampling and analysis from a biological body, whether human or otherwise, using the own natural motions of the said body, said system comprising an integrated unit that comprises: (a) a lancing assembly, (b) an actuator operable to drive movement of said lancing assembly into piercing relation to the biological body at a lancing site thereon, (c) a sample interface holder configured to support a sample interface in a position receiving a fluid sample from the lancing site, and (d) circuitry connected to the sample interface holder to enable sample analysis.

In detailed embodiments, the proposed apparatus consists of rotational and translational actuator components which work together to convert the forces exerted by normal mammalian movement into the power needed to penetrate the skin with a lancet either directly or indirectly through the use of further mechanisms to promote consistency of penetrations. One embodiment further comprises an electrically-controlled mechanical locking mechanism that initially blocks the movements of the rotational and translational actuator components. Further description of the present preferred embodiment of the apparatus is illustrated in the appended drawings.

According to another aspect of the invention, there is provided a method of obtaining a fluid sample from a biological body, whether human or otherwise, said method comprising using natural movement of said biological body to trigger a lancing action releasing a fluidic sample from said biological body.

### BRIEF DESCRIPTION OF FIGURES

FIG. 1a is a cross-sectional side view of the preferred embodiment of the present invention.

FIG. 1b shows a north-east isometric of the apparatus from FIG. 1a.

FIG. 1c is a rear view of the apparatus from FIG. 1a.

FIG. 2a shows the preferred embodiment from FIG. 1a in a locked position.

FIG. 2b shows the apparatus of FIG. 1a in an actuated position.

FIG. 3 shows the apparatus of FIG. 1a integrated in a shoe sole.

FIG. 4a illustrates an alternative test strip position

FIG. 4b reveals an alternative arrangement of multiple test strips

FIG. 5 illustrates the pressure gradient realized in normal walking motions

### DETAILED DESCRIPTION OF THE PREFERRED EMBODIMENTS

For the purposes of promoting an understanding of the principles of this invention, reference will now be made to the embodiments illustrated in the figures and the specific terminology will be used to describe these embodiments. It will be nonetheless understood that no limitation is intended.

The present invention provides a method and its associated apparatus for lancing the skin for the purpose of obtaining and analyzing a whole blood sample. In its preferred embodiment, the invention combines a set of rotational and translational actuation components integrated with a spring-loaded safety lancet as well as an electrically-controlled mechanical locking mechanism that controls the freedom of movement of the set of rotational and translational actuation components. The actuation components are capable of transforming a force exerted on the rotational element into translational movement of the lancet assembly approximately orthogonal to the intended skin's surface. This integrated unit is useful in combination with other devices which preform complementary functions such as collecting, analyzing or testing the blood.

In contrast to the prior art, the present invention introduces an actuator capable of converting the forces exerted by natural mammalian movements, such as, but not limited to, walking, into horizontal translation of the lancet assembly driven by the rotational element. The developed actuation system can (a) harness the energy produced by natural mammalian movements to automatically trigger the skin lancing system; and (b) apply an oscillating pressure to the body to accelerate blood flow from capillary vessels to the skin surface via the wound created by lancing the skin. The proposed actuation method reduces and even eliminates the significant difficulties associated with housing the components of a wearable blood sampling device due to typical space and energy constraints.

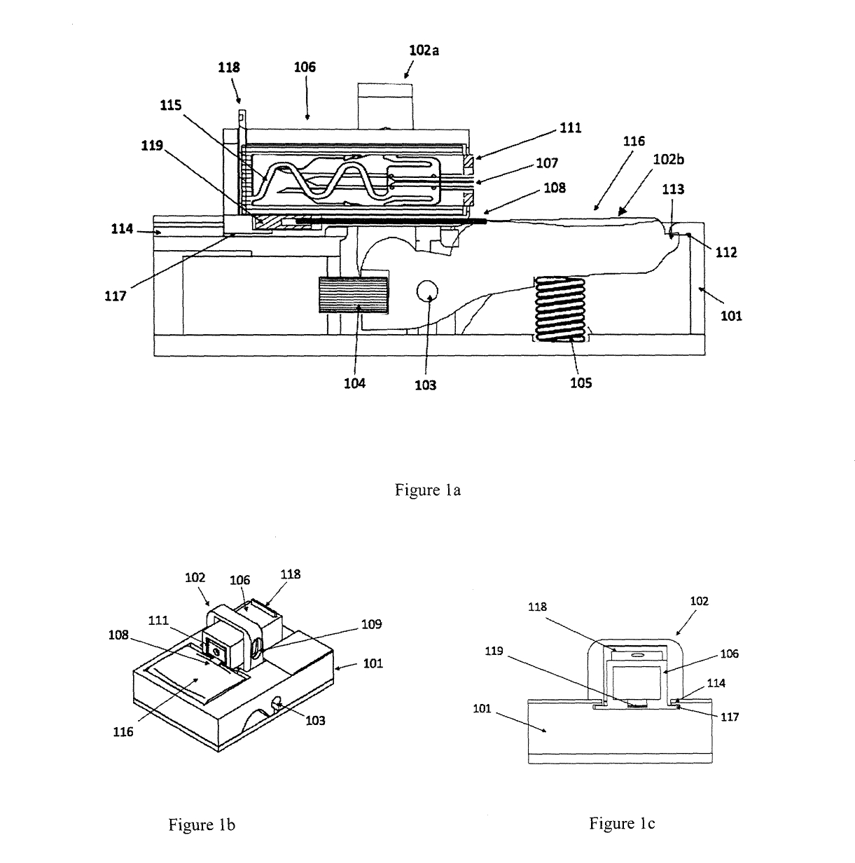

In the preferred embodiment illustrated in FIGS. 1a, 1b and 1c an integrated apparatus unit 100 comprises a body 101 which includes internal structure for supporting the components of the actuation mechanism. Unit 100 may also include means related to blood testing which are described hereafter. Referring to FIG. 1a in detail, a cross-sectional view of the integrated unit 100 is depicted. Unit 100 comprises body 101, rotational element 102, pin 103, locking mechanism 104, return spring 105, sliding element 106, spring-loaded lancet assembly 107, and test strip 108. As described in further detail below, the rotational element, pin, return spring and sliding element cooperatively form an actuator by which the spring-loaded lancet assembly is actuable by natural body movement of a human or other biological body.

The body 101 can be fabricated from a biocompatible hard plastic or metal. The body 101 acts as the framework in which all other components of the actuator are housed and/or are coupled to. The body 101 also features a guiding channel 114 which will be discussed in further details hereafter.

The rotational element 102 can be fabricated from any biocompatible hard plastic or metal. The rotational element has a first lever-like member 102a situated internally of the body 101 and pivotally coupled to the body via pin 103 so that the rotational element 102 has freedom of rotation about the longitudinal axis of pin 103. A second saddle-like member 102b of the rotational element is affixed to and stands upward from the first lever-like member 102a on a side of the pin 103 opposite the return spring 105, and protrudes externally of the body and embraces over the slide element 106 and the spring-loaded lancet assembly 107 carried thereby. The second member 102b of the rotational element engages with the sliding element 106 via a pair of slot joints on opposite sides of the sliding element 106. One of these slot joints can be seen at 109 in FIG. 1b, wherein an upright slot in a respective upright part of the second saddle-like member 102b of the rotational element is slidably engaged by a pin protruding from the side of the sliding element. Via these slots joints 109, the angular displacement of the rotational element 102 about the axis of pin 103 is translated into horizontal displacement of the sliding element 106 in a longitudinal direction parallel to the microneedle of the lancet carried thereby. This particular joint design is, however, not meant to be limiting for converting the rotational force to horizontal force. The sustainable angular displacement of the rotating element is approximately 30 degrees.

The locking mechanism 104 can be implemented using a solenoid as illustrated, but this is not meant to be a limiting method for restricting the movement of rotational element 102. The locking mechanism is responsible for the initial prevention of the movement of the rotational and translational components (i.e. rotational element 102 and slide element 106) of the actuator. It is released in order to initiate the actuation process as described hereafter. The locking mechanism 104 can thus be controlled electronically using an onboard microcontroller of the unit 100 to allow for autonomous actuation. As shown in FIG. 1a, the plunger of the solenoid, when extended, engages with an end of the first member 102a of the rotational element 102 on the side of the pin 103 opposite the return spring 105.

The return spring 105 can be fabricated from metal or plastic as known in the art. The purpose of the return spring 105 is to provide a force which opposes the effects of gravity on the rotational element 102 so that the device is oriented in the initial default position illustrated in FIG. 1a when no external forces are applied. In this illustration, the spring 105 is a coiled compression spring but this is not meant to be considered limiting. The spring is restricted in how far it can expand by coupling of a lip 112 of the body with a corresponding lip 113 on the first member 102of the rotational element 102 at a proximal tip thereof situated opposite the locking mechanism 104. In a the initial default position of the rotational element 102, the proximal tip thereof is in a raised condition placing the lip 113 of the rotational element in abutting contact with the lip 112 of the body 101, while the opposing distal end of the rotational element is in a lowered condition aligned with the solenoid plunger for engagement thereby to lock the rotational element in this position.

The sliding element 106 can be fabricated from any biocompatible hard plastic or metal. The purpose of the sliding element 106 is to hold the spring-loaded lancet assembly 107, and to move the spring-loaded lancet assembly 107 towards a user's toe in the longitudinal direction along a horizontal plane as the result of a force exerted on the rotational element 102 by said user's toe. As described before, the sliding element 106 is coupled with the rotational element 102 via the sliding-pin joints 109. The sliding element 106 also has a dove tail which is coupled with the guiding channel 114 of the body 101 to restrict the movement of the sliding element 106 generated by the translation of the torque on the rotational element 102 to linear translational displacement along a horizontal plane approximately orthogonal to the skin at the tip of the user's toe.

The lancet assembly 107 can be fabricated from any biocompatible material such as stainless steel, titanium, as well as many other suitable materials known in the art. In the preferred embodiment, the lancet of the assembly is driven by a spring 115, but this is not meant to be limiting. This spring is pre-loaded (compressed) and is released by depression of a button 111, as known in the art. The reason the preferred embodiment utilizes a spring-loaded lancet is to promote consistency in actuation parameters. Preferably, the lancet 107 is sufficiently sharpened to make an incision through the skin surface and reach the underlying capillary vessels in order to extract sufficient volumes of blood. In the illustrated embodiment, the spring-loaded lancet is shown to be a separate disposable entity however this is not meant to be considered limiting.

FIG. 1a also depicts a disposable test strip 108 which has a microfluidic blood sample interface at the end closest to the skin. The blood sample interface forms a wall micro-structure that is able to attract a blood sample to enter the channel via capillary action further aided by the oscillating pressure generated by natural mammalian movements which will be described hereafter. As known in the art, the blood testing interface reacts with specific molecules in the blood sample (for example, which is not meant to be considered limiting, blood glucose molecules) releasing electrons that under electrical bias form an electric current through internal conductive wires in the test strip 108 flows to multiple conductive pins at the other end. In the preferred embodiment the test strip 108 is inserted into a groove between the spring-loaded lancet assembly 107 and the bottom of the sliding element 106 in an orientation such that its blood testing interface opening is parallel to the skin of the lancing site at the tip of the user's toe. In the illustrated example, the opening of the blood testing interface thus opens upwardly to receive blood falling from the tip of the user's toe. The test strip is held by a test strip holder 119. The test strip holder connects to electrodes on the test strip allowing the produced current to be conducted to further signal amplification and conditioning circuitry for analysis of the blood sample. The position of the test strip 108 and the test strip holder 117 are not meant to be considered limiting and other locations close to the skin penetration site can be utilized, some of which will be discussed hereafter.

Now referring to FIG. 1b there is shown an isometric north-east view of the integrated unit 100. The figure illustrates the relative locations of the push button 111 of the spring-loaded lancet assembly 107, the test strip 108, and a proximal end 116 of the first member 102a of the rotating element 102. The proximal end 116, except for the lip 113 at the tip thereof, is exposed to the exterior of the body 101 through an upper opening therein in order to accommodate receipt of a user's toe on this proximal end 116 of the rotational element 102. The downward vertical force exerted on the proximal end 116 of the rotational element 102 generates a torque on the rotational element 102 that is further translated into horizontal force and displacement of the sliding element 106 in the forward or proximal direction. Effectively, the actuator is capable of using the vertical force and displacement from normal mammalian movements to create a force on, and displacement of, the sliding element 106 in a longitudinal direction approximately orthogonal to the applied force.

Now referring to FIG. 1c, shown is a back view of the apparatus. This figure illustrates the dove tail 117 of the sliding element 106 in relation to the guiding channel 114 of the body 101. As previously discussed, this sliding joint is responsible for maximizing the conversion of the angular displacement of the rotational element 102 into the approximately perpendicular displacement of the sliding element 106.

FIG. 1c also illustrates the sliding door 118 which can be fabricated from any biocompatible hard plastic or metal. The purpose of the door 118 is to allow for replacement of the disposable spring-loaded lancet presented in the preferred embodiment.

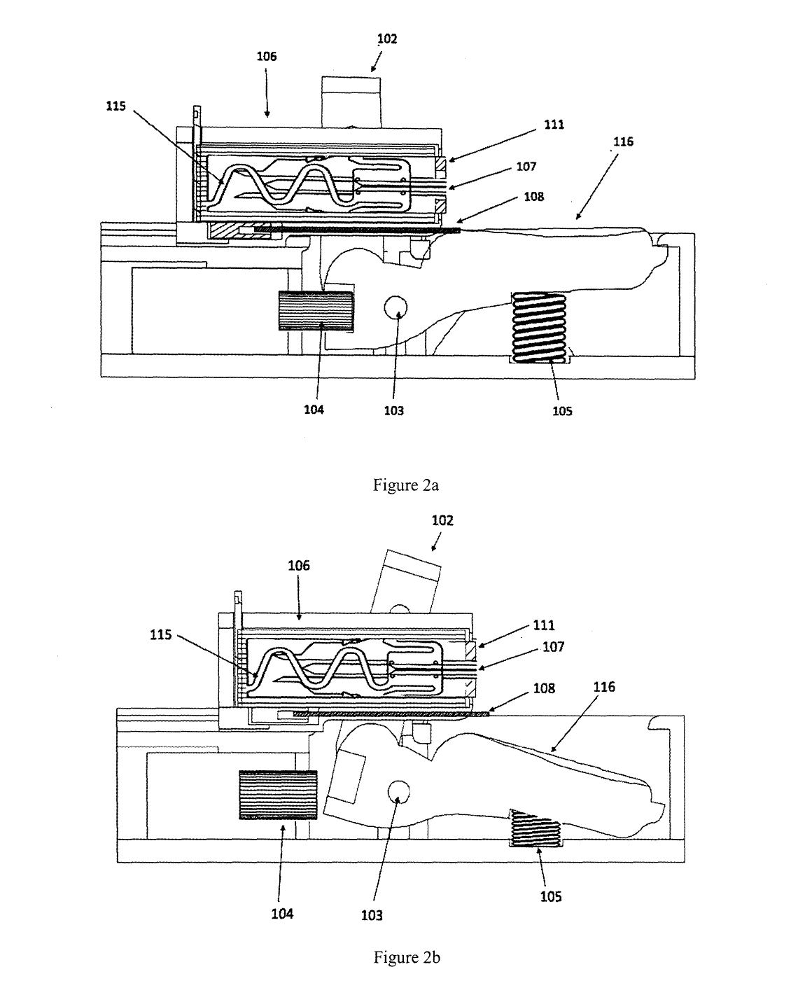

FIGS. 2a and 2b describe the actuation method employed by the preferred embodiment 200. The actuator begins in the position shown in FIG. 2a. This position is stable as the movements of all elements of the actuation system are restricted. Specifically, the rotational element 201 is restricted from angular displacement in one direction by engagement of the locking mechanism 202 in a locked state with the distal end of the rotational element, and restricted from angular movement in the opposite direction by the cooperating lips 112, 113 on the body 101 and the rotational element 102. The force required for the sliding element 106 to move horizontally towards the lancing site on the skin of the user's toe is provided by the torque exerted on the rotational element 102 by said user's toe. By preventing the angular displacement of the rotational element 102, the translational movement of the sliding element 106 is also restricted. Therefore, the lancet assembly 107 is unable to move forward, and so the pressure activated button 111 operable to trigger release of the lancet assembly's spring 115 will not come in contact with the user's skin in this locked position of the actuator.

The actuator is engaged by removing the locking mechanism 104 from its locked state with the rotational element. This is accomplished by supplying adequate power to the push-pull solenoid under the control of the onboard microcontroller, which also carried on the body of the integrated unit along with a battery for powering the microcontroller and locking mechanism solenoid. When the solenoid is activated by the micro-controller, the solenoid plunger retracts into an unlocked state disengaged from the rotational element 102, at which point the rotational element 102 is allowed to rotate freely about the longitudinal axis of pin 103. When a force is exerted on the proximal end of the rotational element, torque is generated on the rotational element 102. The force on the proximal end of the rotational element is supplied by typical mammalian movement such as walking, however this is not meant to be considered limiting. The torque on the rotational element 102 results in clockwise angular displacement of approximately 30 degrees of the rotational element 102. This angular displacement of, and torque on, the rotational element 102 is converted to horizontal translation of, and force exerted on, the sliding element 106. The horizontal translation of the sliding element 106 results in depression of the pressure-activated push button 111 of the spring-loaded lancet 107 as it comes into contact with the intended lancing site on the skin of the user's toe. Depression of the push button 111 triggers the compressed spring 115 to be released, causing the lancet 107 to penetrate the skin with a predetermined force, incision size, and incision depth. The result of these motions is that the actuator is oriented in the position shown in FIG. 2b when the lancet 107 penetrates the skin.

The actuator is returned to is starting orientation shown in FIG. 2a via the return spring 105, which causes counter-clockwise movement of the rotational element 102 about the longitudinal axis of pin 103, when the applied mammalian force is removed from the proximal end of the rotational element 116. In this embodiment, the force is removed as a result of natural oscillations in mammalian movements, however this is not meant to be limiting. The actuator is then locked again via the locking mechanism, which is returned to the position shown in FIG. 2a when the DC current through the solenoid is discontinued by the microcontroller.

The subsequent oscillating exertion of force on the proximal end of the rotational element 116 causes an oscillating pressure within the capillary tissue of the mammal which forces blood to be expelled from the created incision. This blood is then collected by the test strip 108, whose blood sample interface opening is positioned in close proximity to the lancing site to enable this automatic collection of the sample. The blood sample can then be analyzed using methods known in the arts for detecting the presence of concentration of specific molecules such as, which is not meant to be limiting, blood glucose molecules.

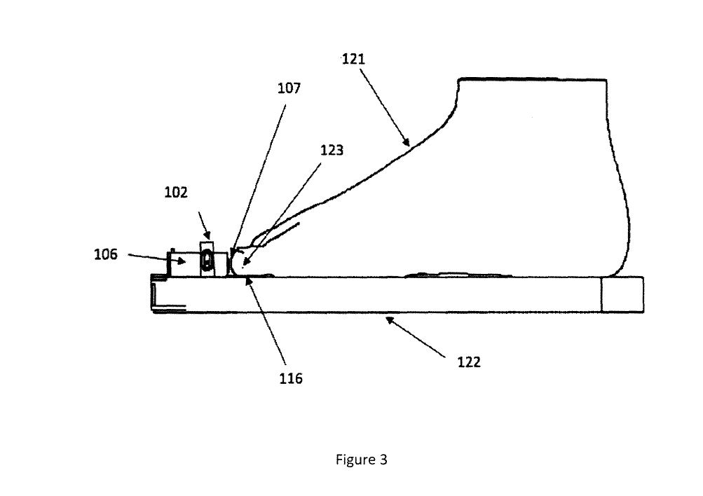

FIG. 3 shows the deployment of the preferred embodiment integrated in the sole 122 of the shoe of a human. This integration is not meant to be limiting but should serve to illustrate the interaction between the actuation system and a human. The foot 121 is an example of a body part which experiences large oscillating forces as the result of walking. The figure illustrates a foot 121 positioned in a fashion so that the medial-most toe 123 is positioned on top of the proximal end of the rotational element 116. In this position, the toe 123 exerts a vertical force on the proximal end of the rotation member 116 with each step. When the locking mechanism (not shown in this particular figure but refer to FIG. 1a, 104) is retracted, the force exerted from the toe 123 depresses the proximal end of the rotational element 116. This rotates the entire rotational element 102 and as described before, provides horizontal movement of the sliding element 106 towards the toe 123. When the button of the spring-loaded lancet assembly 107 is forced against the lancing site at the end of the toe 123, it triggers the lancet 107, resulting in penetration of the skin.

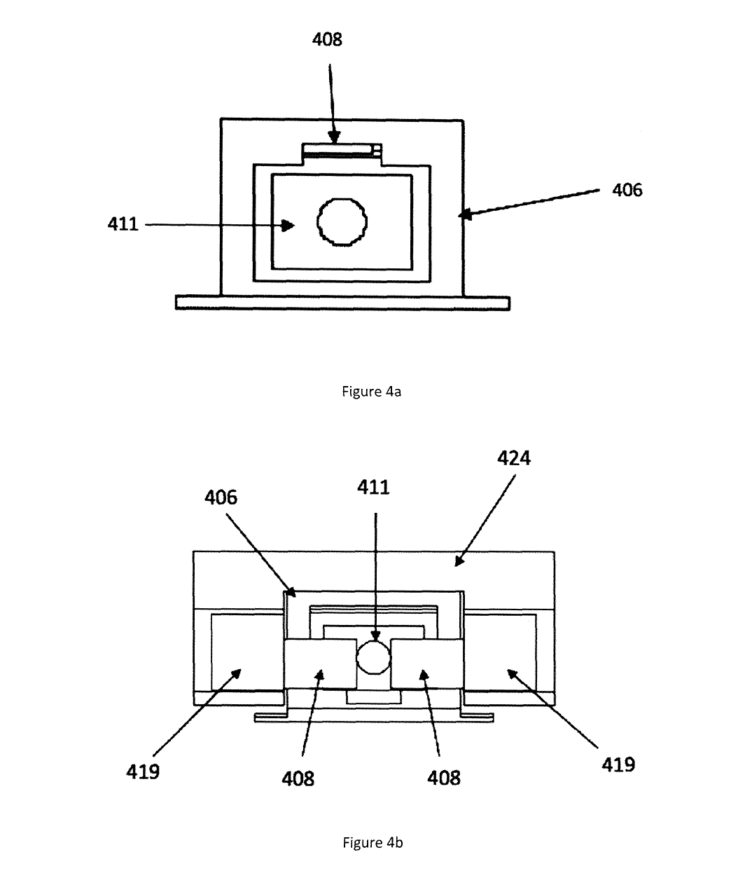

FIGS. 4a and 4b reveal alternative strip positions. FIG. 4a illustrates an embodiment of the lancet holder 406 where the test strip 408 is positioned above the press button 411. The advantage of this positioning is that the motion of the toe depressing the push button results in the toe being relatively lower to its position under normal conditions when it is lanceted. This results in the incision becoming aligned with the opening of the channel of the test strip 408 when the actuator is returned to its initial position.

FIG. 4b shows the positioning of two strips 408 on either side of the push button of the lancet 411. The strips are held in place by the two test strip holders 419 which are supported by a collar 424 fitted to the lancet holder 406 design. The advantage of this configuration is that the probability of collecting a sample is increased. Multiplexing the signals is done in software, although this is not meant to be limiting and hardware multiplexing can also be utilized.

In summary, none of the test strip arrangements presented herein are meant to be limiting but rather to provide an insight into the methods which can deliver reliable test results.

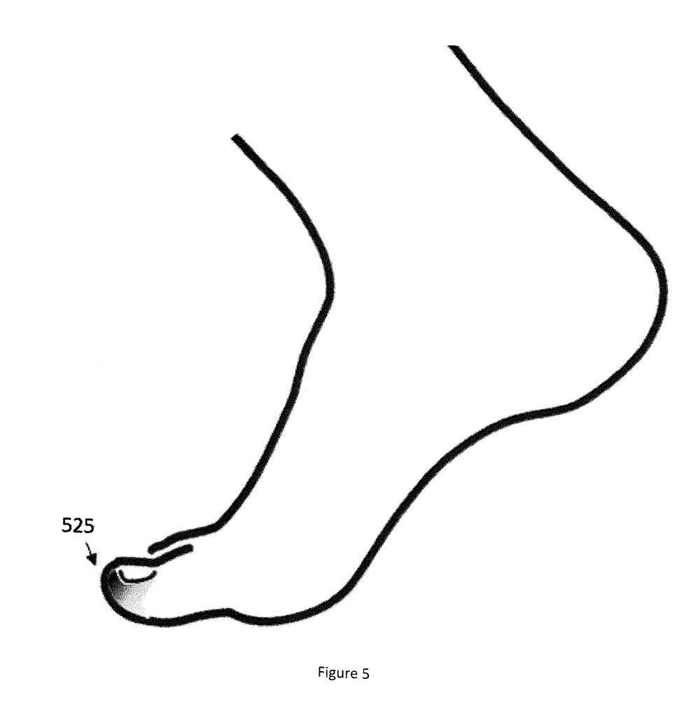

FIG. 5 shows a foot in the “push-off” position experienced during normal human walking cycle. This is included to illustrate the source of the force which is harnessed in this particular embodiment however this is not meant to be considered limiting. The pressure gradient is illustrated by the shaded region 525. This pressure is not only used to trigger the apparatus but is also instrumental in the extraction of adequate volumes of blood through its oscillations during natural walking that contribute to expelling blood out of the created incision and into the test strip setup.

What is claimed is:

1. A system for fluid sampling and analysis from a biological body, whether human or otherwise, using the own natural motions of the said body, said system comprising an integrated unit that comprises: (a) a lancing assembly, (b) an actuator operable to drive movement of said lancing assembly into piercing relation to the biological body at a lancing site thereon, (c) a sample interface holder configured to support a sample interface in a position receiving a fluid sample from the lancing site, and (d) circuitry connected to the sample interface holder to enable sample analysis.

2. The system of claim 1 wherein the actuator is configured to drive transitional displacement of the lancet assembly in parallel relation to a needle direction of said lancet assembly.

3. The system of claim 1 comprising a locking mechanism operable between a locking state preventing or restricting movement of the lancing assembly toward the lancing site, and a release state allowing movement of the lancing assembly toward the lancing site.

4. The system of claim 3 wherein the locking mechanism is electrically operated.

5. The system of 4 wherein the release state of the locking mechanism is triggered by electrical activation thereof.

6. The system of claim 3 wherein the locking mechanism is a mechanical locking mechanism movable between a first position blocking the actuator to achieve the locking state, and a second position retracted from the actuator to achieve the release state.

7. The system of claim 1 comprising at least one electrically controllable mechanism operable to prevent actuation of the actuator.

8. The system of claim 3 wherein the locking mechanism is configured to prevent movement of the lancing assembly in the locking state.

9. The system of claim 8 wherein the locking mechanism is configured to prevent movement of the lancing assembly in the locking state by preventing movement of the actuator.

10. The system of claim 1 wherein the lancing assembly comprises a spring-loaded mechanism configured to release upon the contact of the lacing assembly with the biological body at or exceeding a predetermined pressure threshold.

11. The system of claim 1 incorporated into a footwear sole.

12. A method of obtaining a fluid sample from a biological body, whether human or otherwise, said method comprising using natural movement of said biological body to trigger a lancing action releasing a fluidic sample from said biological body.

13. The method of claim 12 wherein said natural movement is a walking movement, in which the lancing action is triggered by a foot of said biological body.

14. The method of claim 12 comprising using an integrated unit to both trigger said lancing action, and collect said fluidic sample.

15. The method of claim 12 comprising triggering the lancing action and collecting the fluidic sample using the system of claim 1.
